(19) United States
(12) Patent Application Publication
(10) Pub. No.: US 2014/0058234 A1
(43) Pub. Date: Feb. 27, 2014

(54) DEVICE FOR DETECTING BLOOD-OXYGEN LEVEL ASSOCIATED WITH ONE OR MORE MUCOUS MEMBRANE REGIONS

(71) Applicant: Chi Mei Medical Center, Tainan City (TW)

(72) Inventors: Ying-Chieh Su, Tainan City (TW); Jui-Wen Pan, Hsinchu City (TW); Bor-Shyh Lin, Changhua County (TW)

(73) Assignee: Chi Mei Medical Center, Tainan City (TW)

(21) Appl. No.: 13/971,993

(22) Filed: Aug. 21, 2013

(30) Foreign Application Priority Data
Aug. 22, 2012 (TW) 101130454

(51) Int. Cl. A61B 5/1455

(52) U.S. Cl. CPC A61B 5/14551
USPC 600/339; 600/364

(57) ABSTRACT

A device adapted for insertion into one or more of an esophagus, a stomach, an intestine and a colon for detecting a blood-oxygen level associated with at least one mucous membrane region in said one or more of the esophagus, the stomach, the intestine and the colon is disclosed. The device includes a flexible and elongated main body, and a blood oxygen level detecting unit. The blood oxygen level detecting unit includes one or more blood oxygen level detecting modules disposed on the main body and capable of generating one or more signals associated with the blood oxygen level(s) of one or more mucous membrane regions nearby the blood oxygen level detecting module(s).

### CROSS-REFERENCE TO RELATED APPLICATION

This application claims priority of Taiwanese Application No. 101130454, filed on Aug. 22, 2012.

### BACKGROUND OF THE INVENTION

1. Technical field

The disclosure generally relates to a blood oxygen level detector, more particularly to a device for detecting blood oxygen level of internal organ(s).

2. Description of the Related Art

When performing esophagectomy or gastrectomy, it is a norm to reattach the remaining esophagus to the stomach, to reattach the remaining stomach to the intestine or colon, or to reattach the esophagus to the intestine or colon. Referring to FIG. 1 for instance, the portion 101 comprising of part of the esophagus 100 and part of the stomach 103 illustrated by dashed lines is to be removed during esophagectomy, and then the remaining portion 104 of the esophagus 100 is reattached to the remaining portion 107 of the stomach 103.

It is crucial to monitor post-esophagectomy or post-gastrectomy patients for mucosal blood delivery at the anastomosis 105 of the organs. Abnormal mucosal blood delivery may cause ischemia in tissues close to the anastomosis 105 and the closure line 106 of the stomach 103, leading to inflammation or ischemic necrosis of the tissues or associated organs. Generally, medical staff will monitor the patient's vital signs and use drainage tubes to facilitate the detection of postoperative complications. But all of the observation items currently available, such as vital signs and drainage tubes, are delayed parameters. When these parameters show abnormality, the diagnosis is delayed already. In addition, the risks such as forming perforations in the tissues close to the anastomosis 105 and the closure line 106 of the stomach 103 will rise dramatically. There is an urgent need to establish a tool for tissue perfusion monitoring.

### SUMMARY OF THE INVENTION

Some of the embodiments disclosed herein are to provide a device for measuring blood-oxygen level associated with one or more mucous membrane regions of a postoperative patient.

According to the present disclosure, there is provided a device adapted for insertion into one or more of an esophagus, a stomach, an intestine and a colon for detecting a blood-oxygen level associated with one or more mucous membrane regions in said one or more of the esophagus, the stomach, the intestine and the colon. The device comprises a flexible and elongated main body, and one or more blood oxygen level detecting units. Each blood oxygen level detecting unit includes one or more blood oxygen level detecting modules disposed on the main body and capable of generating one of more signals associated with the blood oxygen level(s) of one or more mucous membrane regions nearby the blood oxygen level detecting module(s).

### BRIEF DESCRIPTION OF THE DRAWINGS

Other features and advantages of the present disclosure will become apparent in the following detailed description of the preferred embodiment with reference to the accompanying drawings, of which:

FIG. 1 is schematic diagram for illustrating the procedure of esophagectomy;

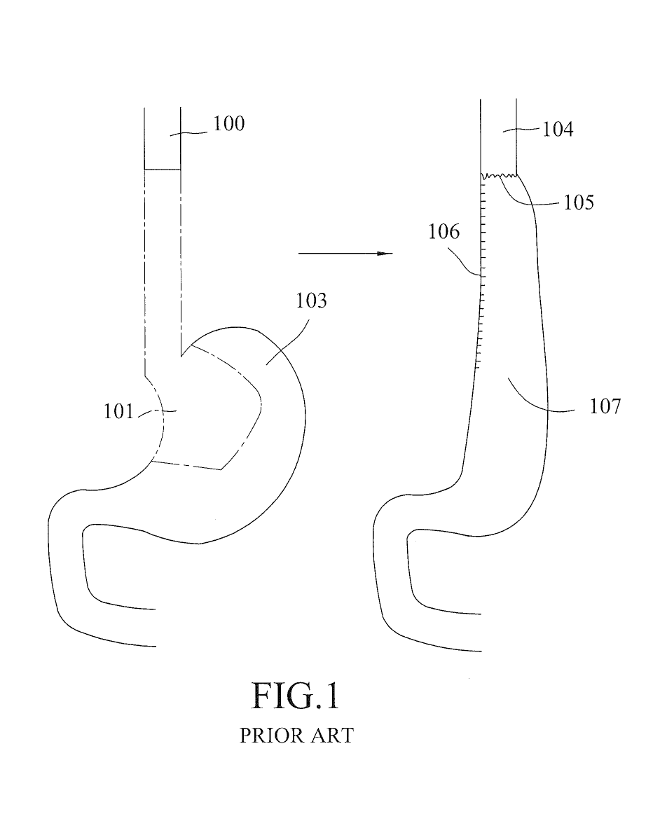

FIG. 2 is a fragmentary perspective schematic view of the first preferred embodiment of a device for detecting a blood-oxygen level associated with one or more mucous membrane regions according to the present disclosure;

FIG. 3 is a block diagram, illustrating a micro-processing unit and blood oxygen level detecting modules of a blood oxygen level detecting unit of the first preferred embodiment;

FIG. 4 is a fragmentary sectional view of the first preferred embodiment;

FIG. 5 is a fragmentary perspective view of the blood oxygen level detecting unit according to the first preferred embodiment;

FIG. 6 is a schematic diagram, illustrating use of the first preferred embodiment on a postoperative patient;

FIG. 7 is a fragmentary perspective schematic view of the second preferred embodiment of a device for detecting a blood-oxygen level associated with one or more mucous membrane regions according to the present disclosure; and

FIG. 8 is a fragmentary perspective view similar to FIG. 5, but according to the third preferred embodiment of a device for detecting a blood-oxygen level associated with one or more mucous membrane regions according to the present disclosure.

### DETAILED DESCRIPTION OF THE PREFERRED EMBODIMENTS

Before the present disclosure is described in greater detail, it should be noted that like elements are denoted by the same reference numerals throughout the disclosure.

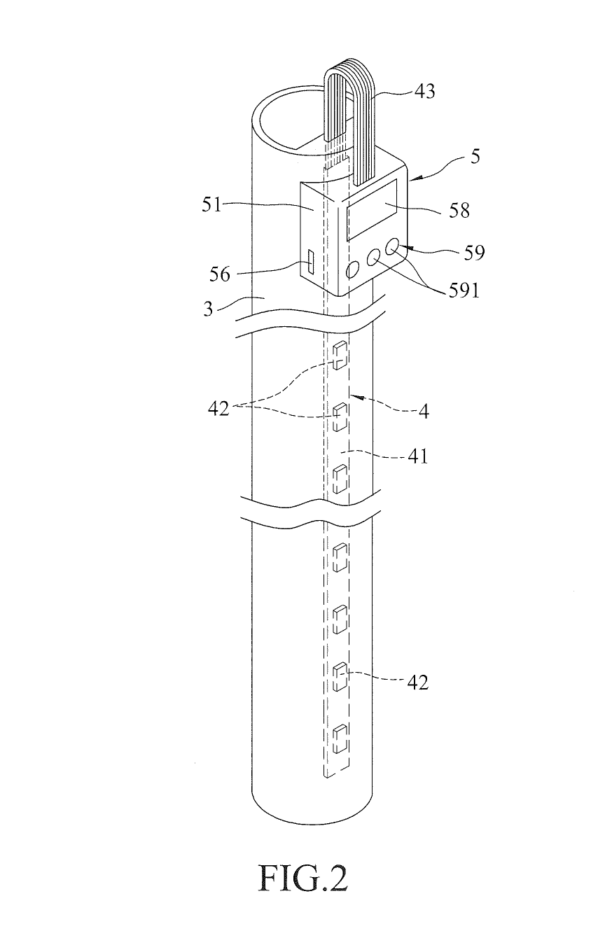

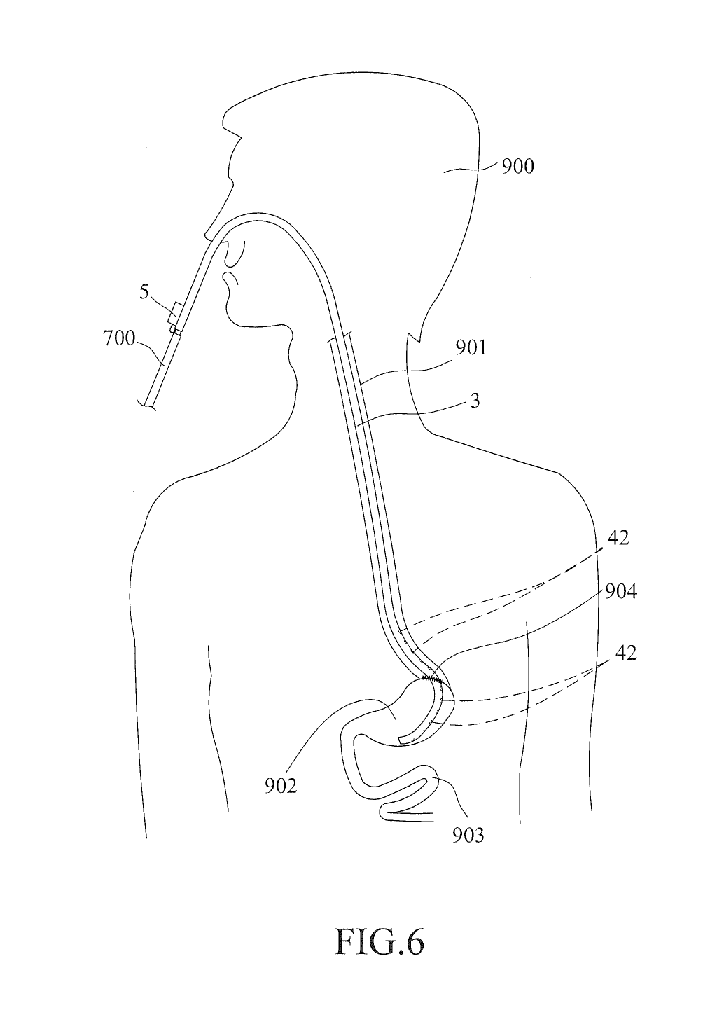

With reference to FIG. 2 and FIG. 6, the first preferred embodiment of a device for detecting a blood-oxygen level associated with one or more mucous membrane regions according to the present disclosure is adapted to be inserted into one or more of an esophagus 901, a stomach 902, an intestine 903 and a colon (not shown) of a patient 900 that has undergone esophagectomy, gastrectomy or a similar operation. The device is able to detect a blood-oxygen level associated with one or more mucous membrane regions in said one or more of the esophagus 901, the stomach 902, the intestine 903 and the colon, preferably near an anastomosis 904 of the organs so as to facilitate evaluation by medical staff of postoperative conditions of the patient 900. Due to the structural configuration of the first preferred embodiment as will be described in greater detail below, the device may be connected to a connecting tube 700 and cooperatively function as a nasogastric tube or a drainage tube. It should be noted herein that the anastomosis 904 may be between the esophagus 901 and the stomach 902, between the esophagus 901 and the intestine 903, between the esophagus 901 and the colon, between the stomach 902 and the intestine 903, between the stomach 902 and the colon, etc. For the purpose of illustration, the following description will be made with reference to a post-gastrectomy patient 900 whose stomach 902 has been partially removed with the remaining portion of the stomach 902 reattached to the esophagus 901.

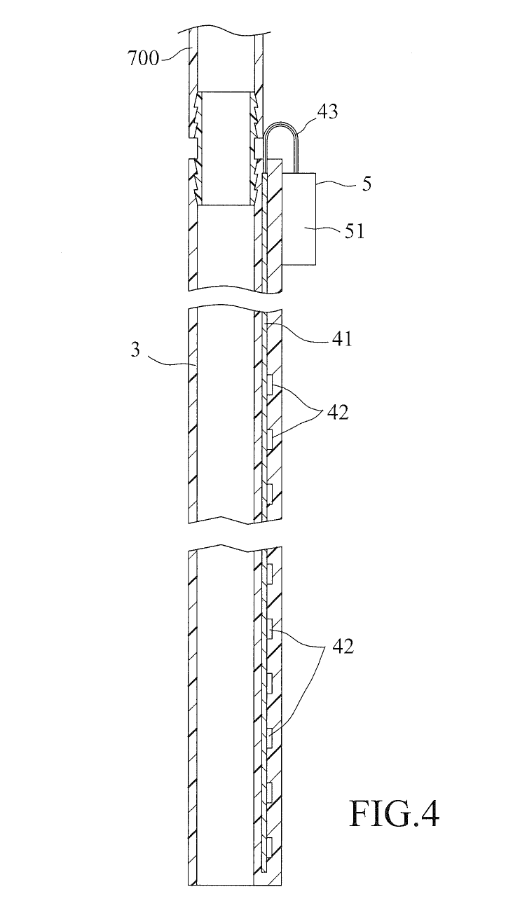

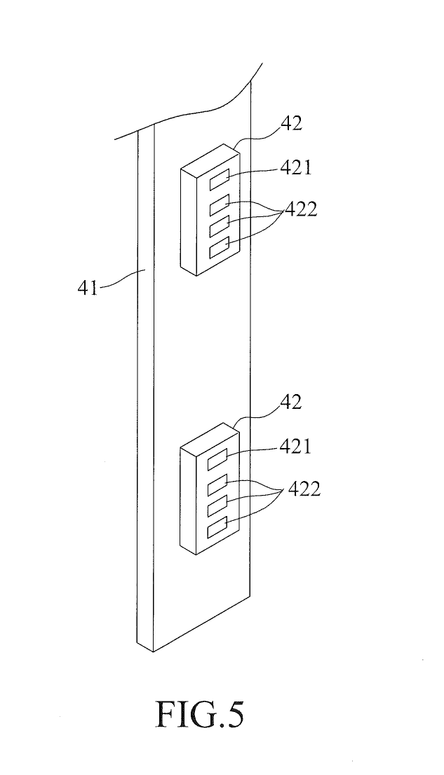

Referring to FIGS. 2 to 5, the device includes a flexible and elongated main body 3, a blood oxygen level detecting unit 4 and a micro-processing unit 5.

The main body 3 may be made from a material that permits passage of near-infrared light and/or red light, such as PVC (polyvinyl chloride) or silicone. In this embodiment, the main body 3 has a tubular configuration so as to be connectable with the connecting tube 700 to function as a nasogastric tube or a drainage tube, thereby allowing the device to serve dual functionalities.

The blood oxygen level detecting unit 4 includes a flexible circuit board 41, a plurality of blood oxygen level detecting modules 42 mounted at the same side on the flexible circuit board 41, and a signal line 43 connected electrically to the blood oxygen level detecting modules 42 via said flexible circuit board 41.

The flexible circuit board 41 may be made of a plastic thin film, and may be formed through semiconductor manufacturing techniques with a plurality of conductive traces (not shown) that are electrically connected to the blood oxygen level detecting modules 42. In this embodiment, the flexible circuit board 41 is embedded within the main body 3 along with the blood oxygen level detecting modules 42, with the blood oxygen level detecting modules 42 being spacedly arranged along a longitudinal direction of the main body 3, while the signal line 43 is partially embedded within the main body 3 with an exposed end connected electrically to the micro-processing unit 5. It should be noted herein that the arrangement of the blood oxygen level detecting modules 42 is not to be limited to being longitudinally in other embodiments of the present disclosure.

Each of the blood oxygen level detecting modules 42 is capable of generating one or more signals associated with the blood oxygen level(s) of one or more mucous membrane regions nearby the blood oxygen level detecting module 42. By providing the plurality of the blood oxygen level detecting modules 42, a plurality of signals associated with a plurality of mucous membrane regions respectively nearby the blood oxygen level detecting modules 42 may be generated.

Specifically, each of the blood oxygen level detecting modules 42 includes one or more light emitters 421 capable of emitting near-infrared light and/or red light to the one or more mucous membrane regions nearby the blood oxygen level detecting module 42, and one or more optical sensors 422 capable of sensing the near-infrared light and/or the red light diffused/reflected by the one or more mucous membrane regions nearby the blood oxygen level detecting module 42 so as to generate the one or more signals. In the first preferred embodiment illustrated herein, each blood oxygen level detecting module 42 includes one light emitter 421 and three optical sensors 422 that are arranged spacedly along the longitudinal direction of the main body 3. The signals generated by the optical sensors 422 are transmitted to the micro-processing unit 5 via the flexible circuit board 41 and the signal line 43 for subsequent analysis, which will be described later.

In this embodiment, with the provision of multiple optical sensors 422, measurement of the blood oxygen levels of different mucous membrane regions may be performed, and with the multiple optical sensors 422 arranged in the longitudinal direction of the main body 3, measurement of the blood oxygen levels of different mucous membrane regions at different depths of the patient 900's gastrointestinal tract is possible.

The light emitter 421 may be a laser diode, a light emitting diode (LED), etc., and the optical sensors 422 may each be a silicon photodiode, an avalanche photodiode, a charge-coupled device, etc. Since the technique for measuring the blood oxygen level by sensing near-infrared light and/or red light is well known in the art, further details of the same are omitted herein for the sake of brevity.

The micro-processing unit 5 is disposed outside of the main body 3, and includes a housing 51, and a light emitter driving module 52, a signal receiving module 53, a micro-processing module 54, a storage module 55 and a wireless communication module 57 that are disposed in the housing 51, and an connector module 56, an output module 58 and an operating module 59 that are mounted to and at least partially exposed from the housing 51.

The light emitter driving module 52 is connected electrically to the light emitters 421 of the blood oxygen level detecting modules 42 via the signal line 43 and the flexible circuit board 41, and is capable of driving each of the light emitters 421 to emit the near-infrared light and/or the red light. The signal receiving module 53 is connected electrically to the optical sensors 422 of the blood oxygen level detecting modules 42 via the signal line 43 and the flexible circuit board 41, and is capable of receiving the signals generated by the optical sensors 422. The micro-processing module 54 is electrically connected to the light emitter driving module 52, the signal receiving module 53, the storage module 55, the connector module 56, the wireless communication module 57, the output module 58 and the operating module 59.

The micro-processing module 54 controls operations of the light emitter driving module 52 and the signal receiving module 53, and is programmable to control the manner in which the light emitter driving module 52 drives the light emitters 421, and the manner in which the signal receiving module 53 receives the signals from the optical sensors 422. In other words, the micro-processing module 54 is able to control the light emitter driving module 52 to independently drive the light emitter 421 of a specific one of the blood oxygen level detecting modules 42, and to control the signal receiving module 53 to receive the signal generated by a specific one of the optical sensors 422 of the specific one of the blood oxygen level detecting modules 42. The signal received by the signal receiving module 53 is then transferred to the micro-processing module 54, which analyzes the signal thus received so as to generate blood oxygen level data associated with the blood oxygen level of the mucous membrane region nearby the specific optical sensor 422 of the specific blood oxygen level detecting module 42. The blood oxygen level data is then stored in the storage module 55, and is outputted for user notification by the output module 58, which in this embodiment, is a display screen.

It should be noted herein that the present disclosure is not to be limited in terms of the manner in which the light emitters 421 and the optical sensors 422 are driven. For example, after driving the light emitter 421 of a specific blood oxygen level detecting module 42 to emit the near-infrared light and/or the red light, the optical sensors 422 of the specific blood oxygen level detecting module 42 may be driven individually and sequentially according to the order of their proximity to the light emitter 421.

The connector module 56 is adapted for electrically connecting an external device (not shown), such as a computer, for input of, e.g., instructions for programming the micro-processing module 54, data to be stored in the storage module 55 for future access and/or execution by the micro-processing module 54, etc. The wireless communication module 57 may be controlled by the micro-processing module 54 to be wirelessly connected to a remote device, e.g., remote centralized equipment (not shown), for transmitting wirelessly the blood oxygen level data thereto. The operating module 59 is operable by a user to control the micro-processing module 54 so that user is provided with the control over the light emitter driving module 52, the signal receiving module 53, the storage module 55, the wireless communication module 57 and the output module 58. In this embodiment, the operating module 59 is shown to include several buttons 591.

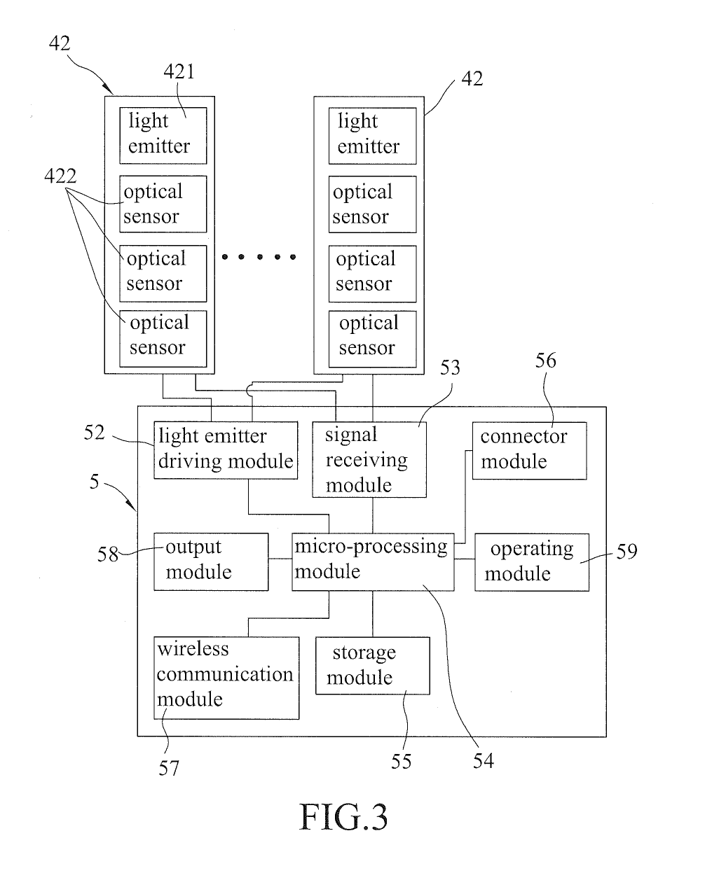

Turning now to FIGS. 3, 5 and 6, to use the device of this disclosure, the end of the main body 3 distal from the micro-processing unit 5 is inserted through the mouth or the nose of the patient 900 into the esophagus 901, and is further extended toward the organs connected by the anastomosis 904, such that the blood oxygen level detecting modules 42 of the blood oxygen level detecting unit 4 is respectively disposed adjacent to the mucous membrane regions nearby the organs. In the example illustrated in FIG. 6, the main body 3 is inserted into the esophagus 901 and the stomach 902 such that the blood oxygen level detecting modules 42 are distributed from the esophagus 901 to the anastomosis 904 and to the stomach 902 so as to be able to measure the blood oxygen levels associated with the mucous membrane regions spreading for a predetermined distance from the esophagus 901 to the anastomosis 904 and to the stomach 902.

After insertion of the main body 3 into the patient 900's body, the end of the main body 3 proximate to the micro-processing unit 5 may be connected to a connecting tube 700 so as to simultaneously function as a nasogastric tube or a drainage tube. Next, a medical staff may operate the operating module 59 to activate the micro-processing module 54 to begin the measurement of the blood oxygen levels, in which the light emitter(s) 421 of the blood oxygen level detecting module(s) 42 is (are) driven to emit the near-infrared light and/or the red light toward the esophagus 901, the anastomosis 904 and/or the stomach 902 for diffusion/reflection thereby and for subsequent sensing by the optical sensor(s) 422 of the blood oxygen level detecting module(s) 42. The micro-processing module 54 then analyzes the signal(s) generated by the optical sensor(s) and generates the blood oxygen level data associated with the desired mucous membrane region(s) nearby the optical sensor(s) 422. The output module 58 may be utilized so as to notify the medical staff in real time of the acquired blood oxygen level data in order for the medical staff to evaluate the mucosal blood delivery at the anastomosis 904 and at the organs (i.e., the esophagus 901 and the stomach 902) connected by the anastomosis 904, thereby assisting in the early discovery of any related abnormal condition.

Since the device also functions as a nasogastric tube or a drainage tube, it can be left inside the patient 900's body to perform long-term monitoring of the blood oxygen levels nearby the anastomosis 904. In addition, without repeated insertion/removal, the device effectively reduces patient discomfort, and promotes the possibility of early detection of leakage and graft failure.

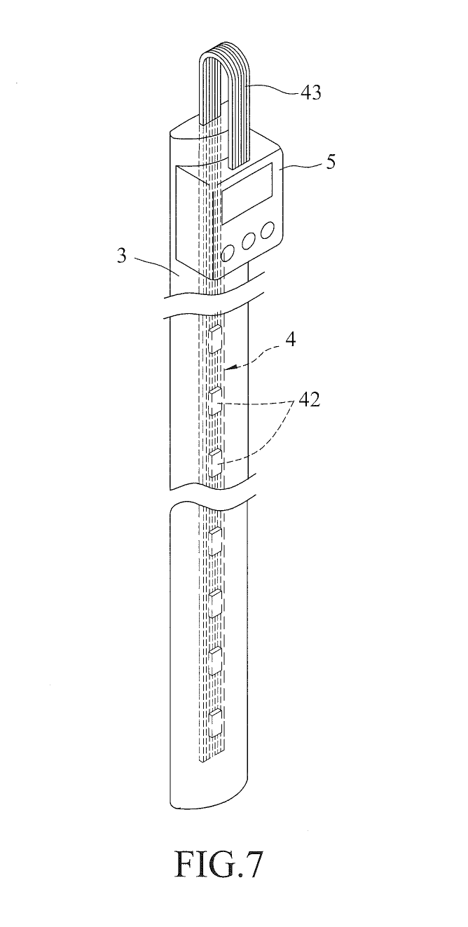

It should be noted herein that while in the first preferred embodiment the main body 3 is designed to be tubular so as to be simultaneously usable as a nasogastric tube or a drainage tube, in practice, the main body 3 of this disclosure is not necessarily tubular, and may be designed into, for example, a solid, thin strip (as illustrated in the second preferred embodiment of FIG. 7).

Furthermore, while in this embodiment the blood oxygen level detecting modules 42 are mounted to and connected electrically to the flexible circuit board 41, and are altogether embedded within the main body 3, and is electrically connected to the micro-processing unit 5 via the signal line 43, which is connected to the flexible circuit board 41, the use of the flexible circuit board 41 is not necessary, and in practice, the blood oxygen level detecting modules 42 may be directly connected to the signal line 43 and embedded within the main body 3 with a segment of the signal line 43 exposed from the main body 3 and connected electrically to the micro-processing unit 5. Alternatively, the blood oxygen level detecting modules 42 may be mounted into an external surface of the main body 3 in a manner so as to be flush with the external surface.

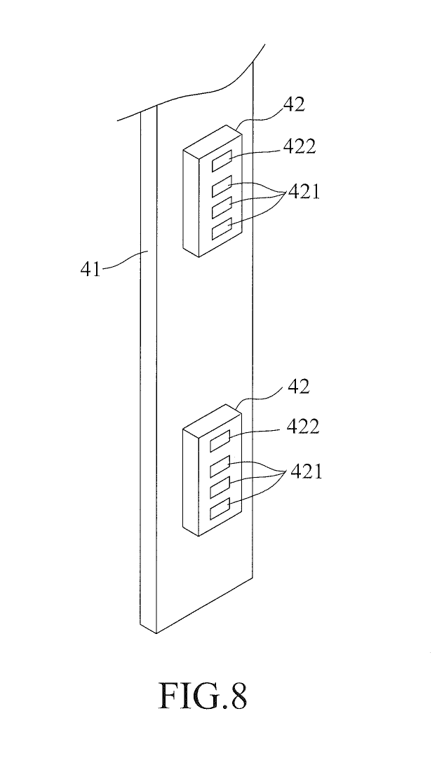

Moreover, each of the multiple blood oxygen level detecting modules 42 of the first preferred embodiment is illustrated as including a light emitter 421 and three optical sensors 422 spaced apart from the light emitter 421 at different intervals. However, in practice, each blood oxygen level detecting module 42 may include a single optical sensor 422 and multiple light emitters 421 spaced apart from the optical sensor 422 at different intervals (as illustrated in the third preferred embodiment of FIG. 8). As such, the device may still be able to measure the blood oxygen levels of mucous membrane regions at different depths in the patient 900's gastrointestinal tract. Alternatively, there may be only one blood oxygen level detecting module 42, which includes one light emitter 421 and one optical sensor 422, for measuring the blood oxygen level of a mucous membrane region at a desired depth in the patient 900's gastrointestinal tract.

It should further be noted herein that the micro-processing unit 5 may be made into a detachable component.

In conclusion, through the design of the flexible and elongated main body 3 bearing one or more blood oxygen level detecting modules 42 thereon, the present disclosure may be inserted and extended into one or more of the esophagus 901, a stomach 902, an intestine 903 and a colon of a post-esophagectomy or post-gastrectomy patient 900 or a patient 900 that has undergone a similar operation to facilitate long-term and real-time monitoring of the blood oxygen level of one or more mucous membrane regions nearby the blood oxygen level detecting module 42 so that medial staff may be able to quickly diagnose the healing/unhealing of the anastomosis 904 of the patient 900 without having the patient 900 experience the uncomfortable and potentially harmful process of repeatedly placing and removing a drainage tube. Additionally, by designing the main body 3 into a tubular structure, the device may also serve as a nasogastric tube or a drainage tube, which is both practical and convenient.

While the present disclosure has been described in connection with what is considered the most practical and preferred embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A device adapted for insertion into one or more of an esophagus, a stomach, an intestine and a colon for detecting a blood-oxygen level associated with at least one mucous membrane region in said one or more of the esophagus, the stomach, the intestine and the colon, said device comprising:
a flexible and elongated main body; and
a blood oxygen level detecting unit including at least one blood oxygen level detecting module that is disposed on said main body and that is capable of generating a signal, which is associated with a blood oxygen level of a mucous membrane region nearby said blood oxygen level detecting module.

2. The device as claimed in claim 1, wherein said blood oxygen level detecting unit further includes a signal line that is connected to said blood oxygen level detecting module for transmitting the signal generated by said blood oxygen level detecting module.

3. The device as claimed in claim 1, wherein said blood oxygen level detecting module includes at least one light emitter that is capable of emitting near-infrared light and/or red light to the mucous membrane region nearby said blood oxygen level detecting module, and at least one optical sensor that is capable of sensing near-infrared light and/or the red light diffused by the mucous membrane region nearby said blood oxygen level detecting module so as to generate the signal.

4. The device as claimed in claim 3, wherein said blood oxygen level detecting module includes a plurality of said light emitters, said light emitters and said optical sensor being arranged spacedly.

5. The device as claimed in claim 4, further comprising a micro-processing unit that is electrically connected to said blood oxygen level detecting unit, that is disposed outside of said main body, and that includes a light emitter driving module capable of driving each of said light emitters of said blood oxygen level detecting module to emit the near-infrared light and/or the red light, a signal receiving module capable of receiving the signal generated by said optical sensor of said blood oxygen level detecting module, and a micro-processing module electrically connected to said light emitter driving module and said signal receiving module for controlling operations thereof, that receives the signal from said signal receiving module, and that analyzes the signal thus received so as to generate blood oxygen level data associated with the blood oxygen level of the mucous membrane region nearby said blood oxygen level detecting module.

6. The device as claimed in claim 5, wherein said micro-processing unit further includes an output module that is connected electrically to said micro-processing module for outputting the blood oxygen level data for user notification.

7. The device as claimed in claim 5, wherein said micro-processing unit further includes a wireless communication module that is connected electrically to said micro-processing module for transmitting wirelessly the blood oxygen level data.

8. The device as claimed in claim 5, wherein said micro-processing unit further includes an operating module that is connected electrically to said micro-processing module and that is operable by a user to control said micro-processing module.

9. The device as claimed in claim 3, wherein said blood oxygen level detecting module includes a plurality of said optical sensors, said light emitter and said optical sensors being arranged spacedly.

10. The device as claimed in claim 9, further comprising a micro-processing unit that is electrically connected to said blood oxygen level detecting unit, that is disposed outside of said main body, and that includes a light emitter driving module capable of driving said light emitter of said blood oxygen level detecting module to emit the near-infrared light and/or the red light, a signal receiving module capable of receiving the signal generated by each of said optical sensors of said blood oxygen level detecting module, and a micro-processing module electrically connected to said light emitter driving module and said signal receiving module for controlling operations thereof, that receives the signal from said signal receiving module, and that analyzes the signal thus received so as to generate blood oxygen level data associated with the blood oxygen level of the mucous membrane region nearby said blood oxygen level detecting module.

11. The device as claimed in claim 10, wherein said micro-processing unit further includes an output module that is connected electrically to said micro-processing module for outputting the blood oxygen level data for user notification.

12. The device as claimed in claim 10, wherein said micro-processing unit further includes a wireless communication module that is connected electrically to said micro-processing module for transmitting wirelessly the blood oxygen level data.

13. The device as claimed in claim 10, wherein said micro-processing unit further includes a storage module that is connected electrically to said micro-processing module for storing the blood oxygen level data.

14. The device as claimed in claim 10, wherein said micro-processing unit further includes an operating module that is connected electrically to said micro-processing module and that is operable by a user to control operation of said micro-processing module.

15. The device as claimed in claim 3, wherein said main body is made from a flexible, light-transmissive material, and said blood oxygen level detecting module of said of said blood oxygen level detecting unit is embedded in said main body.

16. The device as claimed in claim 15, wherein said blood oxygen level detecting unit further includes a flexible circuit board, on which said blood oxygen level detecting module is mounted, and a signal line connected to said blood oxygen level detecting module for transmitting the signal generated by said blood oxygen level detecting module, said circuit board and said blood oxygen level detecting module being embedded within said main body, said signal line being partially embedded within said main body.

17. The device as claimed in claim 1, wherein said main body is tubular.

18. The device as claimed in claim 1, wherein said blood oxygen level detecting unit includes a plurality of said blood oxygen level detecting modules that are spacedly arranged along a longitudinal direction of said main body for generating a plurality of signals associated with a plurality of mucous membrane regions respectively nearby said blood oxygen level detecting modules.

19. The device as claimed in claim 18, wherein each of said blood oxygen level detecting modules includes at least one light emitter that is capable of emitting near-infrared light and/or red light to the mucous membrane region nearby said blood oxygen level detecting module, and at least one optical sensor that is capable of sensing near-infrared light and/or the red light diffused by the mucous membrane region so as to generate the signal.

20. The device as claimed in claim 19, further comprising a micro-processing unit that is electrically connected to said blood oxygen level detecting unit, that is disposed outside of said main body, and that includes a light emitter driving module capable of driving said light emitter of each of said blood oxygen level detecting modules to emit the near-infrared light and/or the red light, a signal receiving module capable of receiving the signal generated by said optical sensor of each of said blood oxygen level detecting modules, and a micro-processing module electrically connected to said light emitter driving module and said signal receiving module for controlling operations thereof, that receives the signal generated by said optical sensor of a selected one of said blood oxygen level detecting modules from said signal receiving module, and that analyzes the signal so as to generate blood oxygen level data associated with the blood oxygen level of the mucous membrane region nearby said optical sensor of the selected one of said blood oxygen level detecting modules.
